(19) United States
(12) Patent Application Publication
(10) Pub. No.: US 2025/0169940 A1
(43) Pub. Date: May 29, 2025

(54) CARTRIDGE INJECTOR SYSTEM WITH IMPROVED LENS BENDING FEATURES FOR USE IN LENS IMPLANTATION

(71) Applicant: VSY BIYOTEKNOLOJI VE ILAC SANAYI ANONIM SIRKETI, Istanbul (TR)

(72) Inventors: Faruk Altan YILDIRIM, Istanbul (TR); Deniz AKI, Istanbul (TR); Bayram PIR, Istanbul (TR); Cem YAPAREL, Kocaeli (TR)

(73) Assignee: VSY BIYOTEKNOLOJI VE ILAC SANAYI ANONIM SIRKETI, Istanbul (TR)

(21) Appl. No.: 18/725,141

(22) PCT Filed: Nov. 8, 2022
(86) PCT No.: PCT/TR2022/051258

§ 371 Date: Jun. 28, 2024

(30) Foreign Application Priority Data
Dec. 28, 2021 (TR) 2021/021433

(51) Int. Cl. A61F 2/16

(52) U.S. Cl. CPC A61F 2/1678; A61F 2/167

(57) ABSTRACT

A cartridge injector system for use in implanting at least one lens into the eye is provided. The system includes at least one cartridge having at least one folding section for positioning the said lens thereon and at least partially bending its ends towards each other, and at least one rotating member capable of bending the said folding section by inserting into the said cartridge. The folding section for bending the lens from its ends in a predetermined manner comprises at least one base and at least one first flap and at least one second flap provided on the opposite sides of the said base in a way that they can be bent to each other and at least one insertion groove for placing the ends of the lens on the said first flap and the said second flap.

### CROSS-REFERENCE TO THE RELATED APPLICATIONS

This application is the national phase entry of International Application No. PCT/TR2022/051258, filed on Nov. 8, 2022, which is based upon and claims priority to Turkish Patent Application No. 2021/021433, filed on Dec. 28, 2021, the entire contents of which are incorporated herein by reference.

### TECHNICAL FIELD

The invention relates to a cartridge injector system for use in implanting at least one lens into the eye, comprising at least one cartridge having at least one folding section for positioning the said lens thereon and at least partially bending its ends towards each other, and at least one rotating member capable of bending the folding section by inserting into the said cartridge.

### BACKGROUND

Intraocular lens therapy is a treatment method used in eye disorders such as cataracts. This method is based on the principle of replacing the natural lenses in the eye with prosthetic lenses produced with special technology. The lens here is a clear structure in the eye. It has very important functions such as refracting the light coming to the eye and transmitting it to the retina and allowing the eye to see objects at every distance.

In some eye disorders, especially cataracts, the lens that has lost its transparency is removed and replaced with an intraocular lens made of collapsible polymer material. The lens insertion process is performed with cartridge injector systems.

Application TR2015/11576 known in the literature relates to cartridge injector systems. This invention has been developed for use in intraocular lens implantation. It allows the lens to pass through the microsurgical incision opened to the eye, holding the lens at its ends in its most basic form, folding it, and thereby implanting it into the eye. The cartridge injector system further comprises an enclosing body that stores the lens in a closed environment and a safety member that limits the rotational movement of the enclosing body.

There are some disadvantages in such applications known in the literature. The most important of these is that the lens is placed on the cartridge section beforehand to push the lens by folding. In the structures of the present art, the lens moves in the cartridge depending on the movement of the cartridge after the lens is placed on the cartridge. As a result of this movement, the lens is positioned in the cartridge and folded in an undesired manner. In this case, the lens can be deformed. Deformation of the lens causes undesired lens-induced accidents in implantation procedures.

All the problems mentioned above have made it necessary to make an innovation in the related technical field as a result.

### SUMMARY

The present invention relates to a cartridge injector system to eliminate the above-mentioned disadvantages and to bring new advantages to the related technical field.

It is an object of the invention to provide a cartridge injector system for use in lens implantation.

Another object of the invention is to provide a cartridge injector system with improved lens bending features.

Another object of the invention is to provide a cartridge injector system with a reduced risk of contamination of the lens.

The present invention is a cartridge injector system for use in implanting at least one lens into the eye, comprising at least one cartridge having at least one folding section for positioning the said lens thereon and at least partially bending its ends towards each other, and at least one rotating member capable of bending the folding section by inserting into the said cartridge, to realize all the objects that are mentioned above and will emerge from the following detailed description. Accordingly, its novelty is that the folding section for bending the lens from its ends in a predetermined manner comprises at least one base and at least one first flap, and at least one second flap provided on the opposite sides of the said base in a way that they can be bent to each other and at least one insertion groove for placing the ends of the lens on the said first flap and the said second flap. Thus, the rotation properties of the lens are improved in the cartridge injector system.

A possible embodiment of the invention is characterized in that it comprises at least one concave form for leaning the lens while rotating in the vicinity of the said insertion grooves. Thus, by leaning the lens on the concave form while rotating, it is ensured that the lens is kept in the insertion groove while rotating.

Another possible embodiment of the invention is characterized in that it comprises at least one jaw in the vicinity of the insertion grooves so that the lens can be held in case of separation from the insertion grooves. Thus, the jaw structure acts as a secondary holder for the lens.

Another possible embodiment of the invention is characterized in that it comprises at least one notch between the first flap and the second flap to improve the rotatability thereof relative to the base. Thus, the lens is rotated without being subjected to undesirable stresses.

Another possible embodiment of the invention is characterized in that the rotating member comprises at least one bending form for bending the said first flap and the said second flap towards each other in the folding section. Thus, the bending of the folding section and accordingly the bending of the lens are improved.

Another possible embodiment of the invention is characterized in that the said bending form comprises at least one first rotating channel for rotating the first flap and at least one second rotating channel for rotating the second flap. Thus, the bending of the folding section and accordingly the bending of the lens are improved.

Another possible embodiment of the invention is characterized in that the bending form comprises at least one pushing protrusion for forcing at least one of the first flap and the second flap to rotate. Thus, the bending of the folding section and accordingly the bending of the lens are improved.

Another possible embodiment of the invention is characterized in that the bending form comprises at least one locking ridge. Thus, it is ensured that the first flap and the second flap are leaned against each other.

Another possible embodiment of the invention is characterized in that the cartridge comprises at least one flange. Thus, rigidity is provided between the discharge end and the folding sections of the cartridge.

Another possible embodiment of the invention is characterized in that it comprises at least one nozzle protrusion on the side of the said flange facing the folding section. Thus, it is ensured that the first flap and the second flap can be rotated towards each other in a predetermined amount.

Another possible embodiment of the invention is characterized in that it comprises at least one flap receiving protrusion provided on at least one of the first flap and the second flap to be leaned against the said nozzle protrusion. Thus, when the first flap and the second flap are leaned on the nozzle protrusion, it is ensured that a stepless opening structure is formed in the interior.

Another possible embodiment of the invention is characterized in that the cartridge comprises at least one channel. Thus, the amount of friction of the lens to the cartridge is reduced.

### BRIEF DESCRIPTION OF THE DRAWINGS

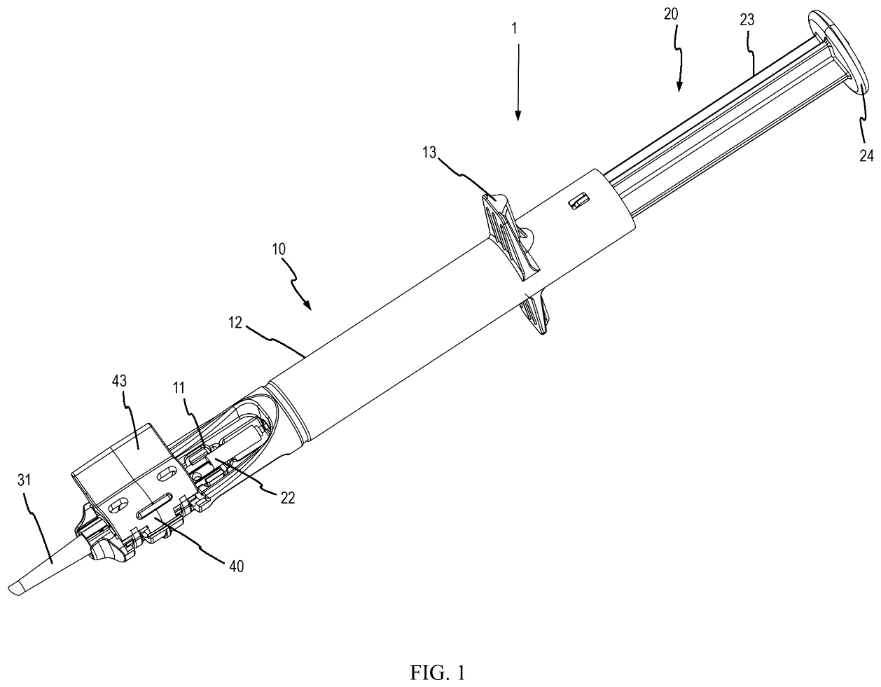

A representative perspective view of the cartridge injector system of the invention is given in FIG. 1.

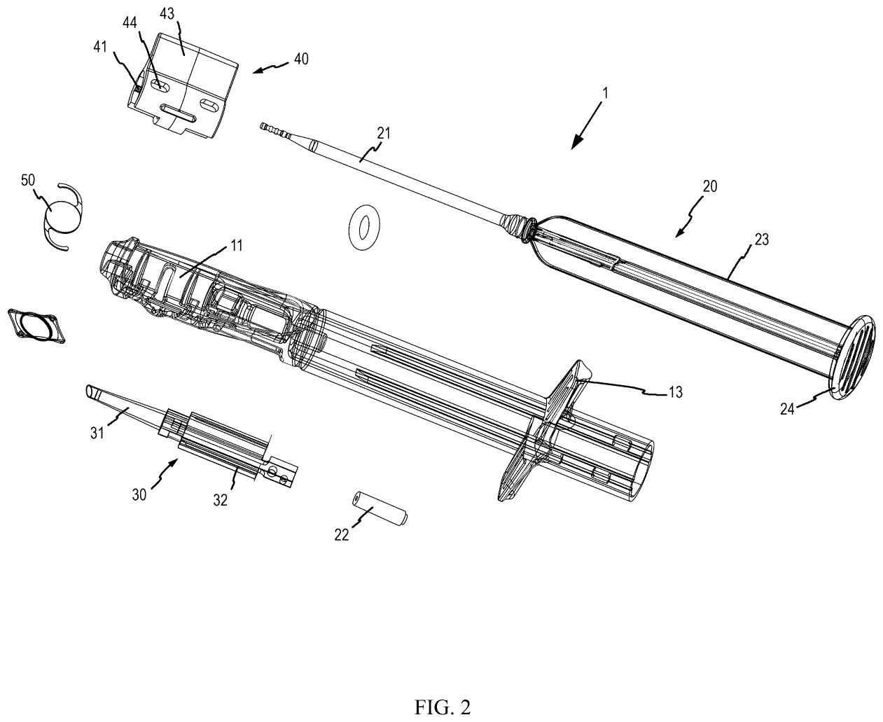

A representative exploded view of the cartridge injector system of the invention is given in FIG. 2.

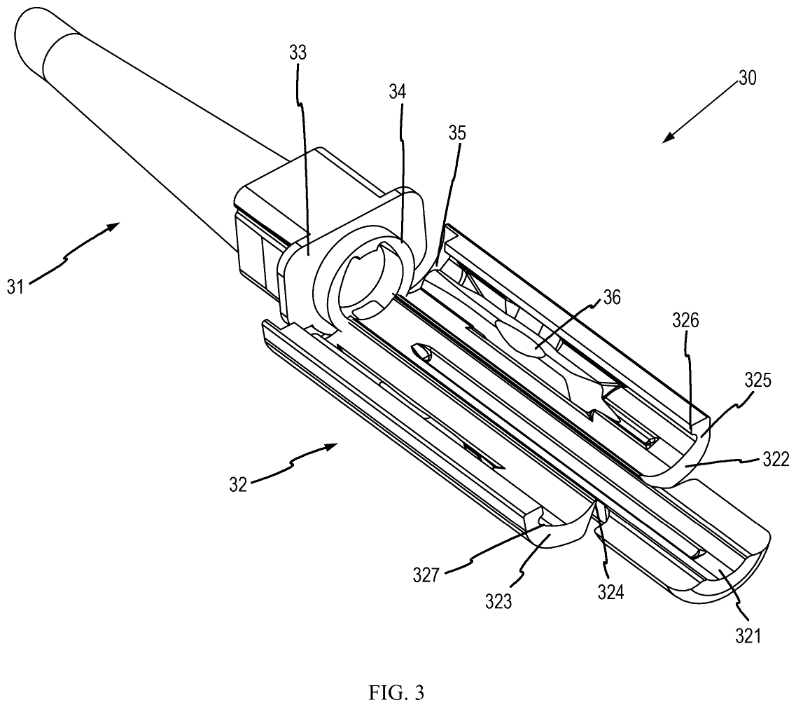

A representative perspective view of the cartridge in the cartridge injector system of the invention is given in FIG. 3.

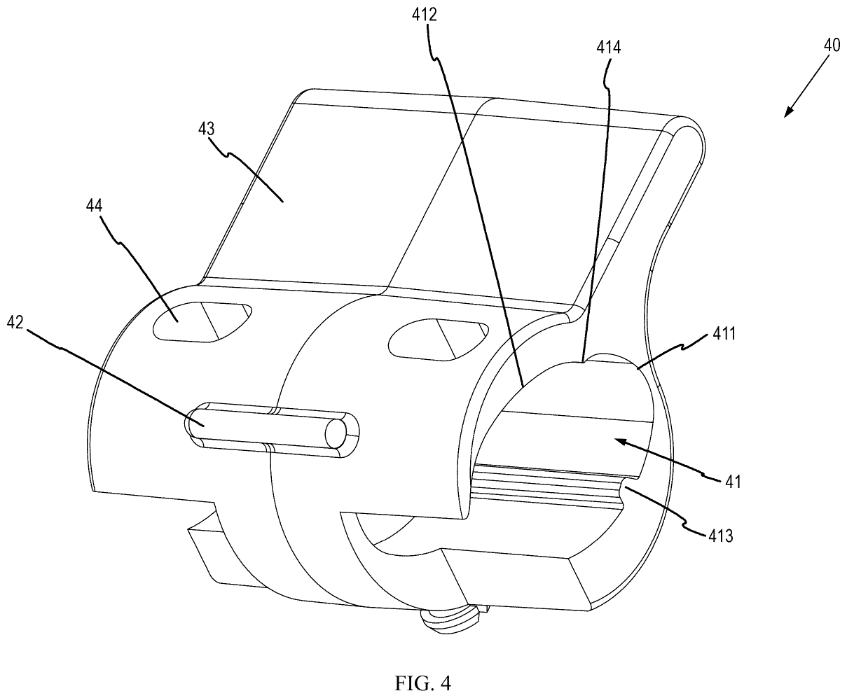

A representative perspective view of the rotating member in the cartridge injector system of the invention is given in FIG. 4.

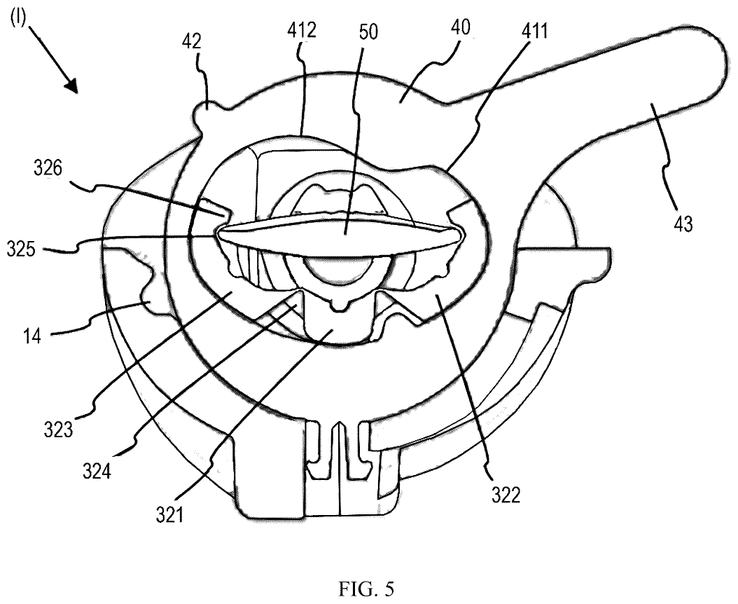

A representative frontal view of the cartridge injector system of the invention in the open position is given in FIG. 5.

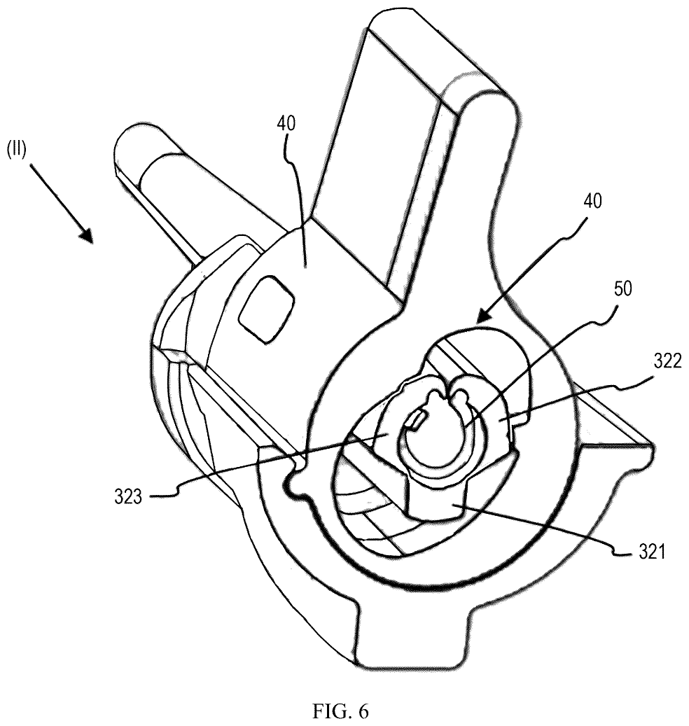

A representative frontal view of the cartridge injector system of the invention in the closed position is given in FIG. 6.

### DETAILED DESCRIPTION OF THE EMBODIMENTS

The subject of the invention is explained with examples that do not have any limiting effect only for a better understanding of the subject in this detailed description.

Representative views of the cartridge injector system (1) of the invention are given in FIGS. 1 and 2. Accordingly, the inventive cartridge injector system (1) is configured to provide at least partial folding of the lens (50) in intraocular implantation procedures. The cartridge injector system (1) is used for placing the artificial lens (50) instead of the natural lens (50) in cases where the eye lens (50) is deformed, such as the cataract. There are various components on the cartridge injector system (1) to perform these operations and they work in sync with each other.

A representative perspective view of the cartridge (30) in the cartridge injector system (1) of the invention is given in FIG. 3. Accordingly, there is at least one cartridge (30) on the cartridge injector system (1). The said cartridge (30) allows the lens (50) placed thereon to be held and folded. There is at least one folding section (32) and at least one discharge end (31) on the cartridge (30). The said folding section (32) allows the lens (50) to be at least partially folded. The said discharge end (31) allows the folded lens (50) in the folding section (32) to be discharged out of the cartridge injector system (1). The discharge end (31) is essentially a tubular outlet provided in a cylindrical and shrinking form to do this. Thanks to the cylindrical and shrinking form of the discharge end (31), the folded lens (50) is discharged without allowing it to be opened from its ends.

There is an end section on the folding section (32) of the cartridge (30). These are at least one base (321), at least one first flap (322), and at least one second flap (323). The said base (321), the said first flap (322), and the said second flap (323) are positioned in the direction of the extension of the cartridge (30). The base (321) is the stationary section of the cartridge (30). The first flap (322) and the second flap (323) are located on opposite sides of the base (321) and are at least partially rotated towards each other. The said first flap (322) and the said second flap (323) may have at least one notch (324) at the joints with the base (321) so that they can have rotational freedom relative to the base (321). The said notch (324) is a shrinking form provided to increase the stretching ability of the first flap (322) and the second flap (323) at the joints with the base (321). Thus, the first flap (322) and the second flap (323) are rotated in a controlled manner.

There is at least one insertion groove (325) on the first flap (322) and the second flap (323). The said insertion grooves (325) are provided essentially symmetrically on the sides of the first flap (322) and the second flap (323) facing each other. The insertion grooves (325) allow the lens (50) to rest on the first flap (322) and the second flap (323). The lens (50) is fixed on the first flap (322) and the second flap (323) at its opposite ends. The insertion groove (325) for this is essentially a channel-shaped structure provided on the first flap (322) and the second flap (323). The dimensions of the insertion groove (325) may be dimensioned according to the dimensions of the lens (50) to be fixed thereon.

There is at least one jaw (326) structure on the outward-facing sides of the insertion grooves (325) on the first flap (322) and the second flap (323). Thanks to the said jaw (326) structure, the grip of the lens (50) is improved. If the lens (50) is separated from the insertion groove (325) during the bending, it is held by the jaw section (326) and the desired rotation process of the lens (50) is completed.

There is at least one concave form (327) on the inward-facing side of the insertion grooves (325) on the first flap (322) and the second flap (323). The said concave forms (327) allow the lens (50) to be gripped from its outer surface during rotation and held stable during rotation. This provides a symmetrical grip of the lens (50) with the mutual provision of two concave forms (327).

There is at least one flange (33) on the side of the discharge end (31) facing the folding section (32) on the cartridge (30). The said flange (33) is essentially a form in which the discharge end (31) is expanded. Thanks to the form it has, the flange (33) provides rigidity and accordingly linearity between the discharge end (31) and the folding section (32). There is at least one nozzle protrusion (34) on the side of the flange (33) facing the folding section (32). The said nozzle protrusion (34) is a ring-shaped protrusion extending essentially from the flange (33) to the folding section (32). The nozzle protrusion (34) is positioned concentrically with the center of the first flap (322) and the second flap (323) when closed. The first flap (322) and the second flap (323) are associated with the nozzle protrusion (34). There is at least one flap receiving protrusion (35) on at least one of the first flap (322) and the second flap (323). The said flap receiving protrusion (35) is preferably provided on both the first flap (322) and the second flap (323). Thanks to the movement of bending the first flap (322) and the second flap (323) towards each other, the flap receiving protrusion (35) is leaned against the nozzle protrusion (34). Thus, the rotational freedoms of the first flap (322) and the second flap (323) are limited, preventing the lens from bending incorrectly inside. The flap receiving protrusion (35) is provided such that it is essentially recessed on the first flap (322) and the second flap (323). Thus, by leaning the flap receiving protrusion (35) against the nozzle protrusion (34), a continuous hole is formed in the inner section of the cartridge (30) and the desired movement of the lens (50) is contributed. In addition, there is at least one channel (36) structure on the cartridge (30). The structure of the said channel (36) is provided in an inwardly bent manner to reduce the amount of friction of the lens (50) to the cartridge (30). In addition, the liquid given in the channel (36) to increase lubricity can be filled and friction can be minimized.

The lens (50) is placed on the cartridge (30) in at least one open position (I) in which it is the widest open. Then, the first flap (322) and the second flap (323) of the cartridge (30) are bent towards each other and the lens (50) is also folded. There is at least one rotating member (40) on the cartridge injector system (1) to perform the bending of the cartridge (30). The cartridge (30) is inserted into the rotating member (40) and the lens (50) is bent by rotating the rotating member (40) at least partially around itself. To do this, the rotating member (40) has a hollow structure. The cartridge (30) may be inserted into this hollow structure. Thereby, the bending of the lens (50) in the cartridge (30) can be adjusted ready for use. For this, the rotating member (40) can be configured to be displaced between an open position (I) and a closed position (II). To do this, there is a unique bending form (41) on the side of the rotating member (40) facing the cartridge (30). The said bending form (41) performs the forming process by stretching the first flap (322) and the second flap (323) of the cartridge (30) as the rotating member (40) is displaced between the two positions.

There is at least one first rotating channel (411) and at least one second rotating channel (412) on the bending form (41). The said first rotating channel (411) is concave to rotate the first flap (322), and the second rotating channel (412) is concave to rotate the second flap (323). The first rotating channel (411) and the second rotating channel (412) on the bending form (41) are intertwined. Thereby, both the first flap (322) and the second flap (323) are rotated during the movement of the rotating member (40) between the open position (I) and the closed position (II). The first rotating channel (411) is provided with a narrower diameter than the second rotating channel (412).

There is at least one pushing protrusion (413) on the bending form (41). The said pushing protrusion (413) is positioned on the inward-facing side of the bending form (41) such that it corresponds to at least one of the notches (324) on the cartridge (30). The pushing protrusion (413) rotates at least one of the first flap (322) and the second flap (323) relative to the other during the movement of the rotating member (40) from the open position (I) to the closed position (II). Preferably, the pushing protrusion (413) rotates the first flap (322) toward the second flap (323). Thereby, rotation problems in the first flap (322) are prevented due to the narrow diameter of the first rotating channel (411).

There is at least one locking ridge (414) on the bending form (41). The said locking ridge (414) allows the jaws (326) to lean against each other if the rotating member (40) is moved to the closed position (II). With the contact of the jaws (326) to the locking ridge (414), the rotation of the lens (50) is completed.

There is at least one locking protrusion (42) on the rotating member (40). The said locking protrusion (42) allows the rotating member (40) to be kept in the closed position (II) continuously. For this, the locking protrusion (42) is inserted into a groove (14) provided on the body (10) and is held continuously.

There is at least one latch (43) on the rotating member (40). The said latch (43) is a protrusion provided on the rotating member (40) to enable the rotating member (40) to be displaced between the open position (I) and the closed position (II). The latch (43) enables the user to control the rotating member (40) by hand. The lens (50) is not bent while in the open position (I). In the closed position (II), the lens (50) is bent and ready for implantation. There is also at least one hole (44) on the rotating member (40). The said hole (44) essentially allows fluid to pass through to reduce the friction between the lens (50) and the cartridge (30). Thereby, the forming of the lens (50) in the cartridge (30) and its discharge from the cartridge (30) are facilitated. The liquid used to do this can be essentially saline, visco liquid. These liquids are distilled water solutions containing certain amounts of sodium bicarbonate and sodium chloride. It ensures that the lens (50) is lubricated in the cartridge (30).

There is at least one body (10) on the cartridge injector system (1). The said body (10) is the main structure provided for the balanced control of the members on the cartridge injector system (1). For this, the body (10) can be essentially hollow cylindrical. There is at least one rotating slot (11) and at least one slide section (12) on the body (10). These two sections are essentially located adjacent to each other. The rotating member (40) and the cartridge (30) are positioned on the rotating slot (11). At least one piston (20) is positioned in the said slide section (12). The said piston (20) allows the lens (50) in the cartridge (30) to be discharged from the discharge end (31). The piston (20) can at least partially move in the direction of the extension of the slide section (12) to do this.

There is at least one piston body (23) and at least one pushing end (21) on the piston (20). The said piston body (23) is shaped so that it is close to the inner diameter of the slide section (12) but narrow from the inner diameter. Thus, it is possible to move the piston (20) on the slide section (12). The pushing end (21) extends between the slide section (12) and the rotating slot (11) of the body (10). The pushing end (21) acts as a pusher by contacting the lens (50) in the cartridge (30). For this, at least one cushion (22) may be located on the pushing end (21) in a possible embodiment of the invention. The said cushion (22) prevents and protects the folded lens (50) from being deformed during pushing. For this, the cushion (22) can be made of a soft material compared to the pushing end (21).

There is at least one holding section (13) on the body (10) and at least one pressing section (24) on the piston (20) for moving the piston (20) with respect to the body (10). The said holding section (13) allows fixing the body (10) with the user's index finger and middle finger. The said pressing section (24) allows the piston (20) along the slide section (12) with respect to the fixed body (10).

The cartridge injector system (1) is essentially positioned in a blister pack. The need for a locking system to prevent the rotation of the rotating member (40) in the cartridge injector system (1) is thereby eliminated. POM is preferred as material in the cartridge injector system (1). POM, also known as acetal, polyacetal, and polyformaldehyde, is an engineering thermoplastic used in precision sections that require high rigidity and excellent size stability. In other possible embodiments of the invention, PC, PP, TPE can also be preferred as material in the cartridge injector system (1). Thus, it is ensured that the expected material properties can be obtained from the cartridge injector system (1).

In a possible use of the invention, the lens (50) is placed in the insertion grooves and the cartridge (30) is placed inside the rotating member (40). Saline is delivered through the holes (44). The rotating member (40) is then rotated from the latch (43) to be moved from the open position (I) to the closed position (II). During this process, the lens (50) is shaped by bending the first flap (322) and the second flap (323) towards each other and leaning the flap receiving protrusions (35) against the nozzle protrusion (34). By moving the piston (20) along the slide section (12) of the body (10), it is ensured that the lens (50) is removed from the discharge end (31) of the cartridge (30). Thereby, the lens (50) becomes used in intraocular implantation. The lens (50) accidents are prevented by preventing undesired forming of the lens (50) in the cartridge (30) during rotation with this configuration.

The scope of protection of the invention is specified in the attached claims and cannot be limited to those explained for sampling purposes in this detailed description. It is evident that a person skilled in the art may exhibit similar embodiments in light of the above-mentioned facts without departing apart from the main theme of the invention.

### REFERENCE NUMBERS GIVEN IN THE FIGURES

1 Cartridge Injector System
10 Body
11 Rotating Slot
12 Slide Section
13 Holding Section
14 Groove
20 Piston
21 Pushing End
22 Cushion
23 Piston Body
24 Pressing Section
30 Cartridge
31 Discharge End
32 Folding Section
321 Base
322 First Flap
323 Second Flap
324 Notch
325 Insertion Groove
326 Jaw
327 Concave Form
33 Flange
34 Nozzle Protrusion
35 Flap Receiving Protrusion
36 Channel
40 Rotating Member
41 Bending Form
411 First Rotating Channel
412 Second Rotating Channel
413 Pushing Protrusion
414 Locking Ridge
42 Locking Protrusion
43 Latch
44 Hole
50 Lens
(I) Open Position
(II) Closed Position

What is claimed is:

1. A cartridge injector system for use in an implantation of at least one lens into an eye, comprising
at least one cartridge having at least one folding section for positioning the lens on the folding section and at least partially bending ends of the lens towards each other, and
at least one rotating member allowed for bending the folding section by inserting into the cartridge, wherein
the folding section for bending the lens from the ends of the lens in a predetermined manner comprises at least one base, at least one first flap and at least one second flap provided on opposite sides of the base in a way that the first flap and the second flap are allowed to be bent to each other, and at least one insertion groove for placing the ends of the lens on the first flap and the second flap.

2. The cartridge injector system according to claim 1, wherein the cartridge injector system comprises at least one concave form for leaning the lens while rotating in a vicinity of the insertion grooves.

3. The cartridge injector system according to claim 1, wherein the cartridge injector system comprises at least one jaw in a vicinity of the insertion grooves, wherein the lens is allowed to be held in case of separation from the insertion grooves.

4. The cartridge injector system according to claim 1, wherein the cartridge injector system comprises at least one notch between the first flap and the second flap to improve a rotatability the first flap and the second flap relative to the base.

5. The cartridge injector system according to claim 1, wherein the rotating member comprises at least one bending form for bending the first flap and the second flap towards each other in the folding section.

6. The cartridge injector system according to claim 5, wherein the bending form comprises at least one first rotating channel to rotate the first flap and at least one second rotating channel to rotate the second flap.

7. The cartridge injector system according to claim 5, wherein the bending form comprises at least one pushing protrusion for forcing at least one of the first flap and the second flap to rotate.

8. The cartridge injector system according to claim 5, wherein the bending form comprises at least one locking ridge.

9. The cartridge injector system according to claim 1, wherein the cartridge comprises at least one flange.

10. The cartridge injector system according to claim 1, wherein the cartridge injector system characterized in that; it comprises at least one nozzle protrusion on a side of a flange facing the folding section.

11. The cartridge injector system according to claim 10, wherein the cartridge injector system comprises at least one flap receiving protrusion provided on at least one of the first flap and the second flap to be leaned against the nozzle protrusion.

12. The cartridge injector system according to claim 1, wherein the cartridge comprises at least one channel.
